(19) United States
(12) Patent Application Publication
(10) Pub. No.: US 2015/0305920 A1
(43) Pub. Date: Oct. 29, 2015

(54) METHODS AND SYSTEM TO REDUCE STUTTERING USING VIBRATION DETECTION

(71) Applicant: Meditab Software Inc., Oakland, CA (US)

(72) Inventor: Miteshkumar Ishwarbhai Patel, San Leandro, CA (US)

(73) Assignee: Meditab Software Inc., Oakland, CA (US)

(21) Appl. No.: 14/265,016

(22) Filed: Apr. 29, 2014

(51) Int. Cl. A61F 5/58; H04R 1/10; H04R 1/14; A61B 5/00

(52) U.S. Cl. CPC A61F 5/58; A61B 5/4803; A61B 5/4836; H04R 1/1091; H04R 1/14

(57) ABSTRACT

A system to reducing stuttering includes two devices. A vibration sensing device is worn around the user's neck and detects when a stuttering event is taking place due to the vibrations of the vocal cords. The vibration sensing device transmits a signal to at least one head phone device inserted in the ear of the user. The head phone device plays music into the ear of the user. The music breaks the connection between the sub-conscious mind and the nerves in the vocal cords is broken temporarily. An application executable on a smart device may facilitate this process.

### FIELD OF THE INVENTION

The present invention relates to a system using components to reduce stuttering. More particularly, the present invention relates to the system and its components detecting vibration produced in the vocal chords of a user to reduce stuttering.

### DISCUSSION OF THE RELATED ART

Stuttering is a speech disorder characterized by speech disruptions, such as prolongations of speech sounds, syllables or words, frequent repetitions, or the inability to start a word. The problem probably is not caused by a single issue, but is the result of a combination of factors. More importantly, the factors causing stammering are different for each person. Thus, the solutions to prevent stammering may differ for each person.

Some of physical or biological factors that cause stammering include a family history of stammering, the brain organization of the user, and the coordination of the speaking mechanism within the body. A link may exist between how a child develops speech and language. This link is important. A problem may arise with some minor, often unrecognized, difficulties. An example would be in “word retrieval” or “word finding.” The filing system for vocabulary is not very efficient. The person knows exactly what he or she wants to say, but the exact word to use escapes him or her for a second or two. A person's instinctive or natural rate of talking also may influence whether stammering occurs.

Other factors may be environmental. Environmental factors include family life, a rapid pace of life (commitments, activities, demands, stress), pace of speech, noise or auditory confusion, or pressure to speak. Personality of the person also may have some bearing on stammering. Personality factors include high achievers, sensitivity, perfectionist disorders (or self-critical), and worriers. The level or existence of these factors differs from person to person.

Treatments for those who stutter strive to teach one skills, strategies and behaviors that facilitate oral communication. Current therapies for teens and adults focus on learning ways to minimize stuttering during speech. Conventional methods may include speaking slowly, regulating breathing, or gradually processing from single syllable responses to longer words or more complex sentences. Most of these methods and therapies help address the anxiety for a person who stutters, especially in certain speaking situations.

Drug therapies also exist to help with stammering. The U.S. Food and Drug Administration (FDA) has not approved any drug for the treatment of stuttering. Drugs, however, are approved to treat other health problems, such as epilepsy, anxiety and depression. These drugs have been used to treat stuttering. Side effects, unfortunately, make these drugs difficult to treat stuttering over a long period of time. Further, drug therapy has been largely ineffective in controlling stuttering.

Some people use electronic devices to help control fluency. For example, one device fits into the ear canal, much like a hearing aid. This device digitally replays a slightly altered version of the user's voice into the ear so that it sounds as if he or she is speaking in unison with another person. These devices work on the feedback mechanism.

Many people achieve success through a combination of self-study and therapy. Self-help groups provide a way for people who stutter to find resources and support. Other proposed solutions include relaxation and breathing exercises. These exercises seek to relax and exercise the vocal cords and improve confidence. Unfortunately, these activities take a lot of time and commitment, sometimes years.

Recently, scientists are examining whether implanting electrodes in the brain, such as those used in treating obsessive-compulsive disorder. Most people, however, will not allow the placement of electrodes in their brains.

The most frequently used anti-stuttering devices are based on delayed auditory feedback. The devices are portable, and include some type of headphones or earpieces in order to reproduce the voice of the user with some delay. In some people, electronic devices help improve fluency in a relatively short period of time. Questions remain about how long such effects may last, and whether people are able to easily use these devices in real-work situations.

An example of such a device includes two headphones and at least one microphone that are connected to an amplifier. The device includes a delay circuit in which the signal from the amplifier to one of the headphones is delayed in comparison to the other signal. The undelayed signal is transmitted to one of the user's ears. The undelayed signal is sensed earlier by the dominant half of the brain so that the other half of the brain is not able to induce stuttering.

The headphones are made for permanent use and include a set of miniaturized earpieces of the type inserted into the ear canal. The earpieces and the microphone are connected to a single portable unit for the user depending on his age and intensity of the stuttering problems.

A disadvantage with these types of devices is that they block the ear canal of the user. The auditory feedback is based on the principle that the sound is amplified and fed into the auditory meatus to stimulate the eardrum from the outside, or a traditional air-conducting hearing aid principle. In order to prevent acoustic feedback problems in these devices, the auditory meatus is plugged substantially by a hearing plug or by the hearing aid device. This causes the user a feeling of pressure, discomfort and sometimes eczema. Running ears due to chronic ear inflammations or infections in the auditory canal may result.

Another disadvantage is that the device must be removed when it is not in use. If the device remains in the auditory meatus when the user is listening, then the device blocks sound from the surroundings. Further, a person with stuttering problems usually does not suffer from hearing problems, and does not need a hearing aid.

### SUMMARY OF THE INVENTION

Therefore, there is need for a stuttering prevention device that does not have the above-discussed inconveniences. It is an object of the invention to provide an anti-stuttering device that includes the principles of user-comfort and health. Instead of the traditional devices and methods discussed above, the disclosed embodiments include a pair of head phones, or ear buds, that are placed in a user's ears. The disclosed embodiments also include a vibration sensor worn around the neck or throat by the user.

The disclosed embodiments include a vibration sensor to sense the vibration produced by the vocal cords of the user. The disclosed embodiments activate music via the head phones. The connection between the sub-conscious mind and nerves that connect vocal cords is broken temporarily. Speech may flow uninterrupted.

The sensor according to the disclosed embodiments detects speech or vibrations produced by the vocal cords by measuring acoustic signals through the skin of the speaker. While the signal produced is not typical compared to a normal acoustic microphone, the sensor may be used as a microphone. Signals from the sensor may be used to activate pre-recorded audio songs in the head phones worn by the user.

The head phones play music into the ears of the user. The music may be stored within a memory in the head phones. In other words, a file is stored and accessed in the head phones. Alternatively, the head phones may communicate with an application executing or active on a smart device, such as a smartphone. The application may be downloadable on a variety of systems, such as Android, IOS or Windows. The application then accesses music files stored on the device and transmits the data for the music file to the head phones.

A stuttering reduction system is disclosed. The stuttering reduction system includes a vibration sensing device located proximately to the vocal cords of a user. The vibration sensing device determines vibration within the vocal cords and generates a signal. The stuttering reduction system also includes at least one head phone device to receive the signal and configured to play an audio signal.

A method for reducing stuttering also is disclosed. The method includes detecting vibration in vocal cords using a vibration sensing device. The method also includes determining that the vibration is related to a stuttering event. The method also includes receiving a signal at a head phone device in response to the determination of the stuttering event. The method also includes playing an audio signal using at least one head phone device.

A stutter reduction device configured to receive data from a vibration sensing device and to determine a stuttering event for a user also is disclosed. The stutter reduction device includes a housing and a display. The stutter reduction device also includes a receiver to receive a signal from the vibration sensing device. The stutter reduction device also includes processing circuitry, disposed in the housing, to determine the stuttering event according to the signal and configured to generate an audio signal within a head phone device.

A device also is disclosed. The device is configured to detect vibration in vocal cords using a signal from a vibration sensing device. The device also is configured to determine that the vibration is related to a stuttering event. The device also is configured to transmit an audio signal to a head phone device in response to the determination of the stuttering event.

### BRIEF DESCRIPTION OF THE DRAWINGS

The accompanying drawings are included to provide further understanding of the invention and constitute a part of the specification. The drawings listed below illustrate embodiments of the invention and, together with the description, serve to explain the principles of the invention, as disclosed by the claims and their equivalents.

FIG. 1 illustrates a stuttering reduction system according to the disclosed embodiments.

FIG. 2 illustrates a head phone device according to the disclosed embodiments.

FIG. 3 illustrates a block diagram of the head phone device and the vibration sensing device according to the disclosed embodiments.

FIG. 4 illustrates a flowchart for a process to reduce stuttering according to the disclosed embodiments.

FIG. 5 illustrates another stuttering reduction system according to the disclosed embodiments.

FIG. 6 illustrates a block diagram of a device used to execute an application for reducing stuttering according to the disclosed embodiments.

### DETAILED DESCRIPTION OF THE PREFERRED EMBODIMENTS

Aspects of the invention are disclosed in the accompanying description. Alternate embodiments of the present invention and their equivalents are devised without parting from the spirit or scope of the present invention. It should be noted that like elements disclosed below are indicated by like reference numbers in the drawings.

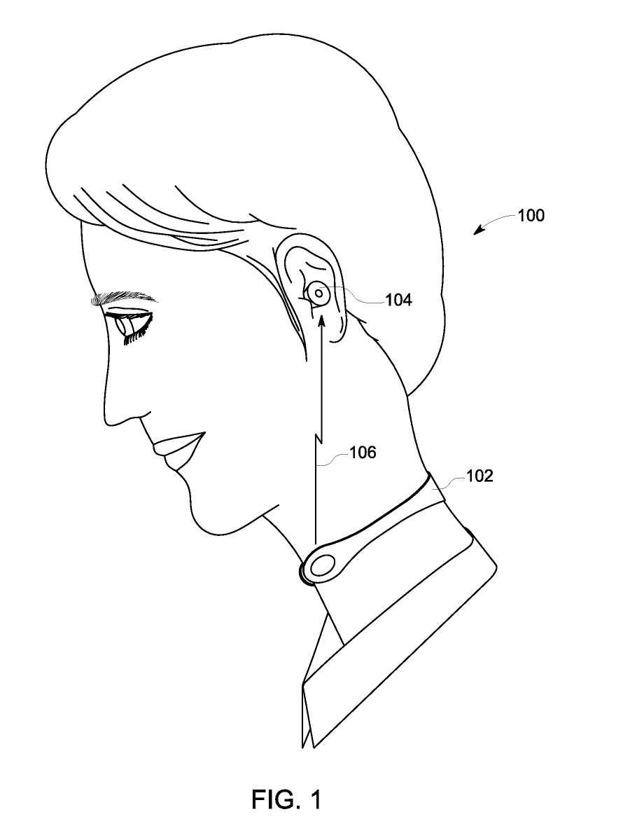

FIG. 1 depicts a stuttering reduction system 100 according to the disclosed embodiments. Stuttering reduction system 100 includes at least two devices. Vibration sensing device 102 may be worn by a user around the neck. Preferably, vibration sensing device 102 rests against the vocal cords of the user. Vibration sensing device 102 senses when the user begins to stutter by the unusual vibration patterns of the vocal cords.

Upon detection of a stuttering event, vibration sensing device 102 transmits a signal 106 to wireless head phone device 104. Head phone device may refer to an ear bud, or any other device capable of playing music or sound into the ear of the user. Although one head phone device 104 is shown in FIG. 1, the user may wear two head phone devices, or one in each ear. Upon receipt of signal 106, head phone device 104 activates music to be played. The music helps by temporarily breaking the connection between the sub-conscious mind and nerves that connect the vocal cords. In other words, the music helps reduce stuttering when it is detected. For some users, the hearing of a song may produce an internal “beat” that helps prevent stuttering, much like a metronome that helps prevent stuttering.

Preferably, head phone device 104 is worn in the ear, and may be hidden from view. Vibration sensing device 102 may appear as a necklace or brooch. The components for each device are housed within, and should not appear unwieldy or obstruct normal activities by the user. The music played into the user's ear may from a certain genre. Preferably, the music is instrumental without many lyrics or no lyrics at all. For example, light jazz music may be played. Classical or ambient music also may be played. The user may select the type of music. As disclosed in greater detail below, music may be played on a smart device or player (such as an MP3 player) and transmitted to head phone device 104.

Vibration sensing device 102 may use a physiological sensor for automatic speech recognition. This sensor detects speech or vibrations produced by the vocal cords by measuring acoustic signals through the skin of the user. By being located close to vocal cords, these signals should be detectable. The sensor is disclosed in greater detail below.

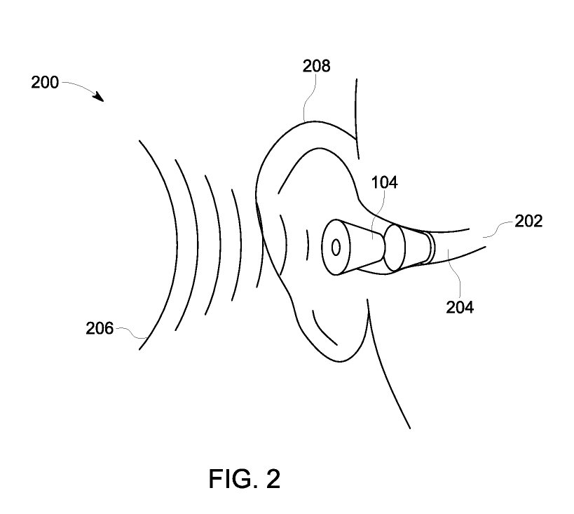

FIG. 2 depicts a head phone device 104 within an ear 208 of a user according to the disclosed embodiments. Ear 208 includes an ear canal 202 and ear drum 204. Head phone device 104 fits snugly within ear canal 202. It should not fall out of ear canal 202, but also head phone device 104 should not totally block the passage within ear canal 202. Head phone device 104 also should not touch ear drum 204.

Upon receipt of signal 106, head phone device 104 transmits music signal, or sound waves, 206. Sound waves 206 may propagate into ear canal 202 to be sensed by ear drum 204. Sound waves 206 also may be transmitted outside ear 208. The user should be able to hear sound waves 206 without unduly external noise. Thus, sound waves 206 may cancel incoming external noise, also known as noise cancelling. The user may only hear the stutter reduction music when a stuttering event occurs.

Preferably, head phone device 104 operates in the frequency range adequate for human hearing. For example, head phone device 104 may operate within the accepted standard range of 20 Hz to 20,000 Hz. This frequency range may be adjustable by the user.

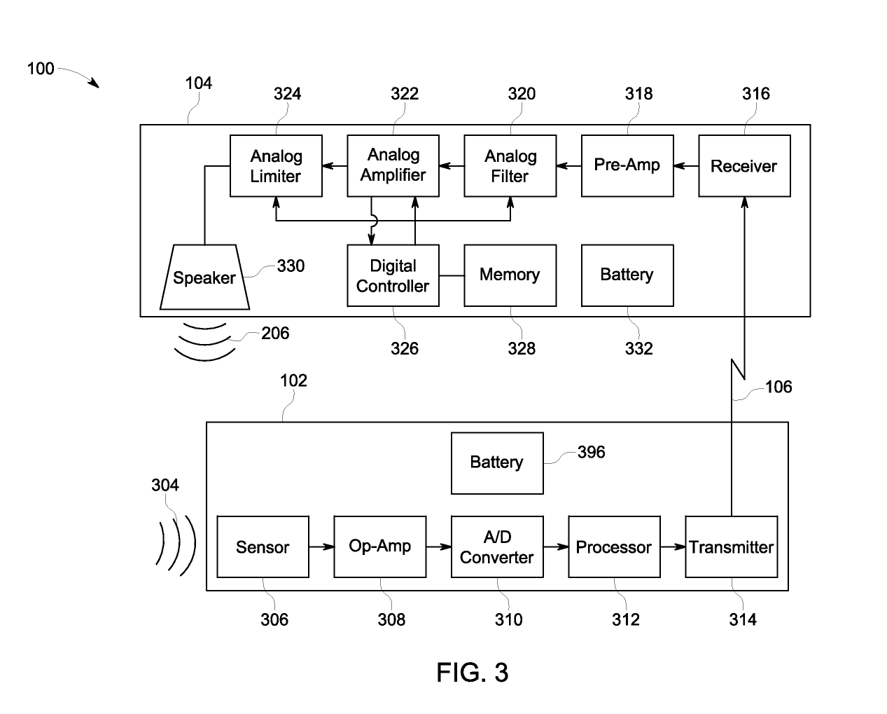

FIG. 3 illustrates a block diagram of head phone device 104 and vibration sensing device 102 according to the disclosed embodiments. System 100 shows the preferred components for use in the devices, though the devices may includes additional components as desired. Further, each component may be a circuit or circuit element that performs the functions disclosed below for each component.

Vibration sensing device 102 is shown as receiving vibration force 304. Preferably, sensor 306 is located close to the vocal cords of the user. Sensor 306 may be a piezoelectric transducer sensor. The piezoelectric sensor uses certain crystals or other materials to transform mechanical energy into electrical output. When a piezoelectric material is subject to a stress or force, it generates an electrical potential or voltage proportional to the magnitude of the force. The voltage induced from the pressure corresponds to that applied pressure.

Thus, piezoelectric sensors may be used to detect single pressure events as well as repetitive events. These properties make this type of transducer preferable as a converter of mechanical energy or force into electric potential. Further, because the modulus of elasticity may be known for the substrate material within the sensor, the unconstrained mass sensor embodiment is allowed to move with vibration, thereby making this type of piezoelectric sensor ideal for detecting vibrations within the vocal cords of the user.

Thus, an electric signal is output by sensor 306 upon detection of vibration force 304. Sometimes, the voltage outputs from piezoelectric sensors may be quite high. Operational amplifier 308 may be used to provide high input impedances to minimize current from potentially high-voltage inputs from sensor 306. Preferably, operational amplifier 308 may be used in a voltage mode. Operational amplifier 308 helps protect analog-to-digital (A/D) converter 310 and processor 312 from potential damage by the current from sensor 306.

A/D converter 310 receives the sensed and corrected signal and converts it into a digital format. A digital signal may be more readily analyzed by processor 312. Processor 312 determines if the signal meets set criteria to be identified as caused by stuttering. Processor 312 may compare the digital signal to stored signal values. These stored signal values may represent patterns that match stuttering vocal characteristics of the user. Using algorithms, processor 312 may determine whether the user is stuttering.

If processor 312 determines the user is stuttering due to the values of vibration force 304, then it sends an activation signal, or command, to transmitter 314. Transmitter 314 then transmits signal 106. Signal 106 may be sent as a short burst over a small range to hearing aid device 104. Transmitter 314 may be set to a certain frequency by the user.

Vibration sensing device 102 may be powered by battery 396. Battery 396 provides power to processor 312 and transmitter 314. A variety of batteries may be used, preferably those used in conjunction with electronics. Alternatively, battery 396 may be rechargeable such that vibration sensing device 102 is connected to a charger to replenish power.

Receiver 316 of head phone device 104 receives signal 106. Receiver 316 may be tuned to the same frequency as transmitter 314. Receiver 316 then sends the signal to pre-amplifier 318 to amplify the signal, especially if it is too weak. Analog filter 320 may filter the signal or attune it as desired. Analog amplifier 322 further amplifies the signal and feeds it into analog limiter 324.

Analog amplifier 322 also feeds the signal to digital controller 326. Digital controller 326 may act as a processor to determine whether music should be played to the user in response to the receive signal 106. If signal 106 is not strong enough, then head phone device 104 may decide not to play music. In other words, digital controller 326 prevents music from being played inadvertently.

If digital controller 326 determines music is to be played, then it accesses a music file from memory 328. Specific songs may be stored in memory 328. Thus, the user may become familiar with the music that signifies stuttering and helpful in breaking the connection between the sub-conscious and nerves of the vocal cords. Several songs may be stored in memory 328.

Digital controller 326 then provides the music file to analog amplifier 322 to play the music by converting the digital information within the file into sound. The sound is sent to analog limiter 324, and onto speaker 330. Speaker 330 produces sound waves 206 that may go into ear 208 of the user and away from the user to provide noise cancellation.

Battery 332 provides power to head phone device 104. Like battery 396, battery 332 may be any type of battery suitable for electronics use. Power is provided to digital controller 326, speaker 330 and receiver 316. The files stored in memory 328 also remain available.

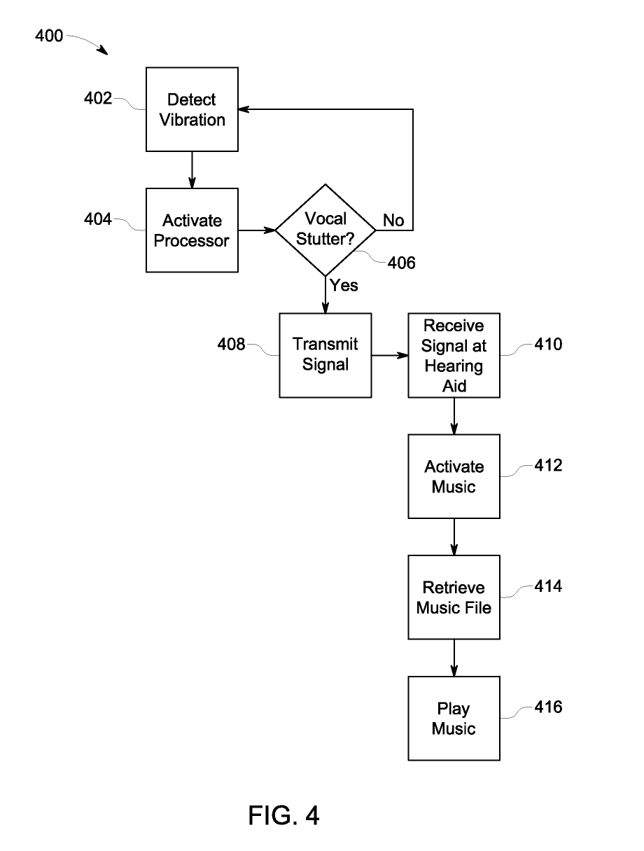

FIG. 4 depicts a flowchart 400 for a process to reduce stuttering according to the disclosed embodiments. The process set forth in FIG. 4 may utilize components disclosed above. Where suitable, the description below references these components for illustrative purposes. The features disclosed by FIG. 4, however, are not limited to the embodiments disclosed above, and vice versa.

Step 402 executes by detecting vibration within the vocal cords of the user. Sensor 306 may pick up the vibrations coming from the vocal cords. Stuttering produces vibrations distinct from normal speech patterns. Step 404 executes by activating processor 312 to determine the status of the detected vibrations. Step 406 executes by determining whether the vibrations are related to a stuttering event by the user. If no, then flowchart 400 returns back to step 402.

If step 406 is yes, then step 408 executes by transmitting signal 106 to head phone device 104. Transmitter 314 receives an instruction from processor 312. Step 410 executes by receiving signal 106 by receiver 316

Step 412 executes by activating the music stored in memory 328 within head phone device 104. Step 414 executes by retrieving the music file from memory 328. Step 416 executes by playing the music through speaker 330. Thus, the music helps prevent continued stuttering by the user as the music breaks the connection and relaxes the user.

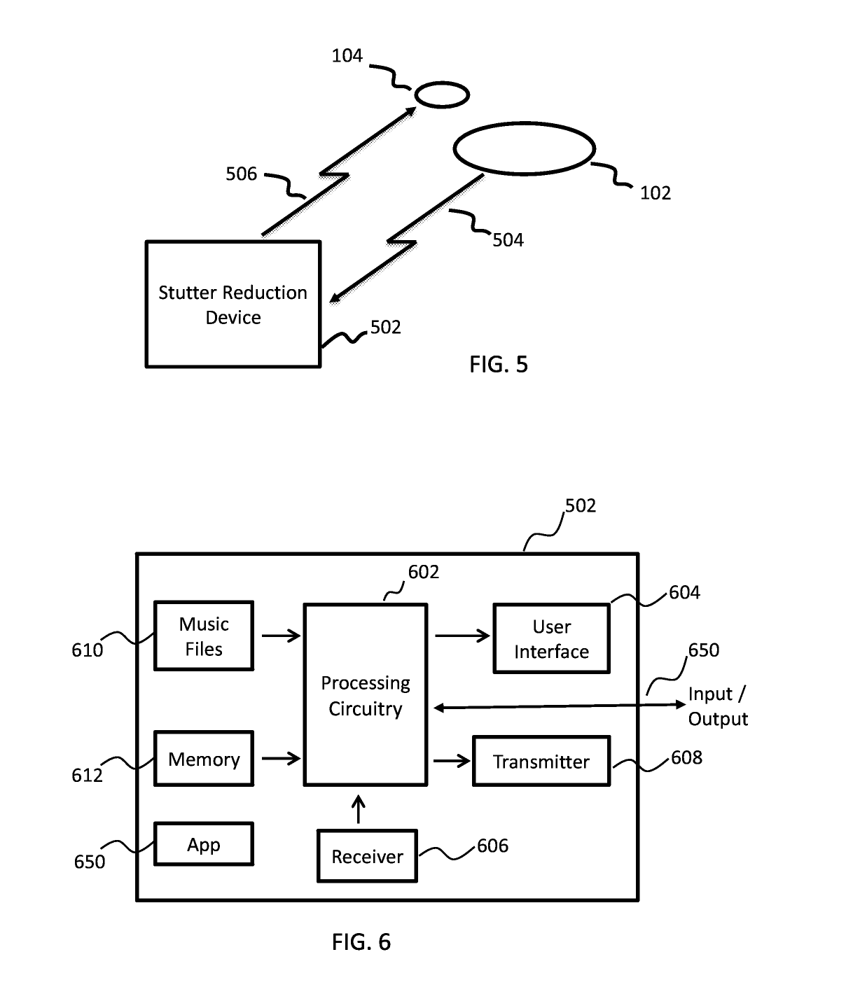

FIG. 5 depicts a stuttering reduction system 500 according to the disclosed embodiments. System 500 differs from system 100 as it includes a smart device 502. Smart device 502 may be a smart phone, smart watch, personal digital assistant, and the like. Smart device 502 stores applications that are executed using the processing capabilities of the device. An application is a program that runs on the device, and, preferably, configures smart device 502 into a special purpose device. In this embodiment, device 502 is configured to a stuttering reduction device.

In this embodiment, vibration sensing device 102 detects vibrations from the vocal cords of the user. When a stuttering event is detected by the sensor in device 102, a signal 504 may be sent to device 502. Stutter reduction device 502 receives signal 504 and determines that the user is stuttering. Stutter reduction device 502 then transmits signal 506 comprising, preferably, music wirelessly to head phone device 104. The application may set the transmission and reception frequencies for the antenna of device 502.

In another embodiment, vibration sensing device 102 merely senses the vibration of the vocal cords and sends these to stutter reduction device 502. Stutter reduction device 502 determines whether the vibrations match a stuttering event. Upon this determination, then stutter reduction device 502 transmits signal 506 to head phone device 104 with the appropriate music to reduce or prevent stuttering. For example, an application executing on device 502 may only access music files selected by the user so that music not really appropriate for stutter reduction, such as heavy metal, is not transmitted to head phone device 104.

FIG. 6 illustrates a block diagram of stutter reduction device 502 used to execute an application 650 for reducing stuttering according to the disclosed embodiments. Stutter reduction device 502 may be a smart phone, smart device, smart watch or the like that includes components used by application 650. Application 650 configures these components to execute the functions disclosed herein. Device 502 is not limited to the components disclosed in FIG. 6, and may include additional components.

Stutter reduction device 502 includes processing circuitry 602. The processing circuitry may be discrete or integrated logic, and/or one or more state machines, processors (suitably programmed) and/or field programmable gate arrays (or combinations thereof); indeed, any circuitry (for example, discrete or integrated logic, state machine(s), special or general purpose processor(s) (suitably programmed) and/or field programmable gate array(s) (or combinations thereof)) now known or later developed may be employed to calculate, determine, assess, estimate and/or determine whether a stuttering event is occurring based on signal 504 or sensor data.

In operation, the processing circuitry may perform or execute one or more applications, routines, programs and/or data structures that implement particular methods, techniques, tasks or operations described and illustrated herein. The functionality of the applications, routines or programs may be combined or distributed. Further, the applications, routines or programs may be implemented by the processing circuitry using any programming language whether now known or later developed, including, for example, assembly, FORTRAN, C, C++, and BASIC, whether compiled or uncompiled code; all of which are intended to fall within the scope of the present invention.

Processing circuitry 602 loads application 650 when instructed, and is configured to execute the instructions associated with application 650. These instructions cause processing circuitry 602 to use the other components shown in FIG. 6. Other components may be used.

Receiver 606 may receive signal 504 from vibration sensing device 102. Processing circuitry 602 may configure receiver 606 to observe transmissions at a set frequency. Connection 605 may provide the input/output capability needed for such observations. For example, connection 605 may be an antenna embedded within stutter reduction device 502.

After receiver 606 receives signal 504, it may convert signal 504 into a digital component for analysis by processing circuitry 602. Processing circuitry 602 may compare the digital data to known stuttering data stored in memory 612. Alternatively, processing circuitry 602 may execute an algorithm to determine if the signal indicates stuttering. Processing circuitry 602 may execute the process disclosed in FIG. 4.

If a stuttering event is determined, then music files 610 may be accessed and played on stutter reduction device 502. A music file is digital data that is converted into a signal containing audible music by processing circuitry 602. For example, processing circuitry may include a media player on device 502. The signal may be sent to transmitter 608 that uses connection 605 to send signal 506 to head phone device 104. Thus, transmitter 608 may convert the audio file to a signal for receiving by head phone device 104.

User interface 604 may provide graphical representations to the user that allows he or she to configure stutter reduction device 502. For example, the user may use user interface 604 to turn application 650 on or off. The user may select which music files 610 are to be played with stuttering is detected. The user also may set how long the music is played and transmitted to head phone device 104.

Thus, stutter reduction device 502 may execute instructions using processing circuitry 602 to execute the functions and actions disclosed above in conjunction with vibration sensing device 102 and head phone device 104. Processing circuitry 602 also may compile information from incoming signals 504.

For example, various analytics may be compiled using processing circuitry 602 and stutter reduction device 502. Application 650 can accumulate data regarding stuttering events, such as how many times he or she has stuttered in a period of time (day, hour, week, etc.) and, also, under what circumstances. Application 650 also may determine for how long music is played before normal vibration of the vocal cords is detected. The application also may determine which songs are effective in reducing stuttering by determining how long before the vocal cords do vibrate. This data may be known as parameters for a stuttering event, or a collection of stuttering events, and stored within memory 612.

This data may be presented to the user via user interface 604. A visual display may provide a dashboard with analytics compiled by application 650 for review by the user. Processing circuitry 602 may be configured to calculate such data and to present it to user interface 604.

It will be apparent to those skilled in the art that various modifications and variations may be made in the disclosed embodiments of the disclosed methods and systems without departing from the spirit or scope of the invention. Thus, it is intended that the present invention covers the modifications and variations of the embodiments disclosed above provided that the modifications and variations come within the scope of any claims and their equivalents.

What is claimed is:

1. A stuttering reduction system comprising:
a vibration sensing device located proximate vocal cords of a user, wherein the vibration sensing device determines vibration within the vocal cords and generates a signal; and
at least one head phone device to receive the signal and configured to play an audio signal.

2. The stuttering reduction system of claim 1, wherein the at least one head phone device is located proximate an ear canal of the user.

3. The stuttering reduction system of claim 1, further comprising a stutter reduction device to transmit the audio signal to the at least one head phone device.

4. The stuttering reduction system of claim 3, wherein the stutter reduction device includes an application stored thereon to determine a stuttering event according to the vibration sensed by the vibration sensing device.

5. The stuttering reduction system of claim 1, wherein the vibration sensing device includes a sensor.

6. The stuttering reduction system of claim 5, wherein the sensor is a piezoelectric sensor.

7. The stuttering reduction system of claim 1, wherein the vibration sensing device is worn around a neck of the user.

8. The stuttering reduction system of claim 1, wherein at least one head phone includes a memory to store a music data file that is converted into the audio signal.

9. A method for reducing stuttering comprising:
detecting vibration in vocal cords using a vibration sensing device;
determining that the vibration is related to a stuttering event;
receiving a signal at a head phone device in response to the determination of the stuttering event; and
playing an audio signal using the at least one head phone device.

10. The method of claim 9, further comprising transmitting the signal from a vibration sensing device.

11. The method of claim 9, further comprising converting a stored music file into the audio signal.

12. The method of claim 11, further comprising retrieving the stored music file from the head phone device.

13. The method of claim 9, further comprising transmitting the signal from a stutter reduction device.

14. The method of claim 9, further comprising storing parameters for the stuttering event in a memory.

15. A stutter reduction device configured to receive data from a vibration sensing device and to determine a stuttering event for a user, the stutter reduction device comprising:
a housing and a display;
a receiver to receive a signal from the vibration sensing device; and
processing circuitry, disposed in the housing, to determine the stuttering event according to the signal and configured to generate an audio signal within a head phone device.

16. The stutter reduction device of claim 15, further comprising a memory to store a music file related to the audio signal, wherein the processing circuitry is configured to convert the music file to the audio signal.

17. The stutter reduction device of claim 16, further comprising the processing circuitry configured to transmit the audio signal to the head phone device.

18. The stutter reduction device of claim 15, further comprising a user interface presented on the display.

19. A device configured to
detect vibration in vocal cords using a signal from a vibration sensing device;
determine that the vibration is related to a stuttering event; and
transmit an audio signal to a head phone device in response to the determination of the stuttering event.

20. The device of claim 19, comprising a memory and processing circuitry.
